(19) United States
(12) Patent Application Publication
(10) Pub. No.: US 2018/0236226 A1
(43) Pub. Date: Aug. 23, 2018

(54) APPARATUS FOR TREATING BLOOD VESSELS IN SKIN

(71) Applicant: Jongju Na, Songpa-Gu (KR)

(72) Inventor: Jongju Na, Songpa-Gu (KR)

(21) Appl. No.: 15/548,765

(22) PCT Filed: Feb. 2, 2016
(86) PCT No.: PCT/KR2016/001140

§ 371 Date: Aug. 3, 2017

(30) Foreign Application Priority Data
Feb. 3, 2015 (KR) 10-2015-0016793
Aug. 13, 2015 (KR) 10-2015-0114641

(51) Int. Cl. A61N 1/32; A61N 1/40; A61N 1/06; A61N 1/36

(52) U.S. Cl. CPC A61N 1/328; A61N 1/36017; A61N 1/06; A61N 1/403

(57) ABSTRACT

A system and method for treating vessel in skin is disclosed. The system may include two or more electrodes that may be inserted in skin to deliver electrical signal to target vessel therein, an electrical signal generator electrically coupled to the plurality of electrodes, and a power supply unit for supplying power to the electrical signal generator. The system and method may be employed to selectively reduce hypervascularity in the dermis or epidermis by generating thermal damage thereto or inducing phagocytosis or apoptosis of vascular cell in order to treat melasma, dermal melasma, hyperpigmentation, hypopigmentation, rosacea, flushing, erythema, or telangiectasia, while lowering the risk of excessive heating of the skin vessel encountered in and reducing the rate of re-occurrence of rebounded vascularization associated with conventional LASER treatments for melasma, dermal melasma, hyperpigmentation, hypopigmentation, rosacea, flushing, erythema, or telangiectasia.

### TECHNICAL FIELD

The present invention relates to an method and system for treating vessels of skin or vessels associated with skin appendage in skin via electric signals. More particularly, to treating blood vessels via electric signals.

### BACKGROUND INFORMATION

While sun exposure, pregnancy, medications, hormonal changes in the body, and genetic factors are thought to affect the development of melasma, dermal melasma, hyperpigmentation, hypopigmentation, rosacea, flushing, erythema, and telangiectasia, the exact mechanisms thereof have yet to be clarified.

Meanwhile, although treatments for hair loss, hair removal, excessive sebaceous gland secretion, excessive sweating, and axillary osmidrosis are increasing in popularity, their efficacy is limited. Research and development of treatment that provide better therapeutic results is warranted.

Some conventional skin treatments employing light energy, LASER energy, and multi-wavelength light energy therapy such as intensive pulsed light (IPL), are not only ineffective at treating targeted vessel at deeper portions of skin, but they also pose an increased risk of damaging skin due to excessive heating thereof during treatment.

Of these conventional therapies, IPL is commonly associated with overheating/burning skin, which may lead to post-inflammatory hyperpigmentation (PIH), as shown in FIG. 1.

### BRIEF DESCRIPTION OF THE DRAWINGS

FIG. 1 is an image depicting potential side effects of conventional treatment prior art devices on skin.

FIG. 2 is a simplified block diagram of a system for generating electrical signals to treat vessel in skin according to various embodiments of the present invention.

FIG. 3 is a simplified illustration of vessel treatment via system delivering electrical signals according to various embodiments of the present invention.

FIGS. 4 to 12 are images depicting the clinical effects of employing a system for delivering electrical signals to vessels in skin during animal experiments according to various embodiments of the present invention.

FIG. 13 is a simplified diagram of a waveform of an electrical signal that may be generated by a system for generating electrical signals to treat vessel in skin according to various embodiments of the present invention.

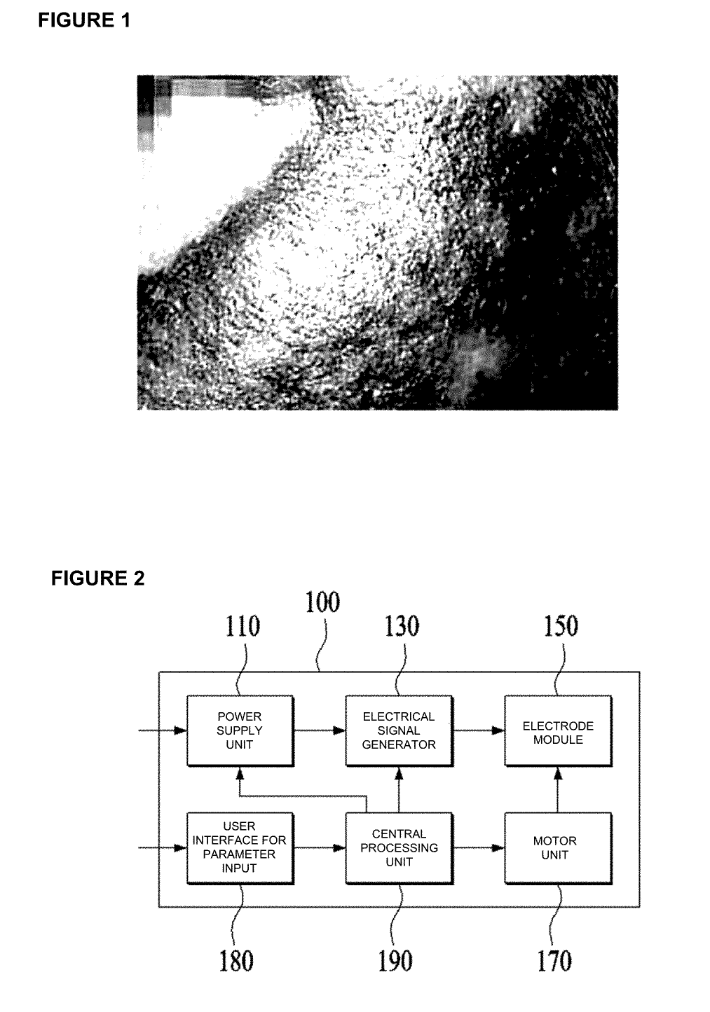

### DETAILED DESCRIPTION

An objective of the present invention is to provide a therapeutic method and system that may be employed to reduce hypervascularity in dermis or epidermis by generating desirable thermal damage thereto or inducing phagocytosis or apoptosis of vascular cells in order to treat melasma, dermal melasma, hyperpigmentation, hypopigmentation, rosacea, flushing, erythema, or telangiectasia while lowering the risk of excessive heating of skin encountered in and reducing the rate of re-occurrence associated with conventional treatments including LASER treatments for melasma, dermal melasma, hyperpigmentation, hypopigmentation, rosacea, flushing, erythema, or telangiectasia.

In accordance with a stated objective, the present invention may provide an system or system for treating vessels, such as blood vessels in skin. The system may including two more electrodes sized to be inserted into skin to deliver electrical signals to a vessel therein to be treated. The system may include an electrical signal generator electrically coupled to the electrodes and a power supply unit for supplying power to the electrical signal generator. In an embodiment, electrical signals delivered to the electrodes may be a repetitive or periodic electrical signal with at least one delay or off duty time.

In an embodiment, a target skin vessel may be positioned between two electrodes. Additionally, electric fields may be formed between the two or more electrodes. The electric field may formed from an alternating current (A.C.) signal applied the electrodes. In addition, some of the electrodes may form bipolar configurations. Additionally, the two or more electrodes may be inserted adjacent to a targeted skin vessel. The application of the system to skin blood vessels may be used to treat one or more symptoms of various skin conditions including melasma, dermal melasma, hyperpigmentation, hypopigmentation, rosacea, flushing, erythema, and telangiectasia. Further, the system may be used to one or more symptoms of various other health conditions by affecting vessels associated with hair loss, hair removal, excessive sebaceous gland secretion, excessive sweating (hyperhydrosis), and axillary osmidrosis.

In an embodiment, an electrical signal delivered through the two or more electrodes into skin may induce a thermal effect on a target skin vessel in a region of skin penetrated by the electrodes. Further, an electrical signal delivered through the electrodes into the skin may induce a thermal effect on an outer layer of a target vessel in region of the skin penetrated by the electrodes. Additionally, a thermal effect may be induced independently around each electrode to the vessel located in region of the skin penetrated by each electrode. Further, a thermal effect may not be generated by an electrical signal conducted in an area between electrodes including an area around a vessel in the area of the skin penetrated by the electrodes. In an embodiment, electrodes may be inserted into dermal layer of the skin.

In addition, an embodiment of the electrical signal generator may include a high-frequency signal generator and generate signals having a frequency is from 0.1 MHz to 100 MHz. Further, an embodiment of the electrical signal generator may include a radio frequency signal generator. In addition, in an embodiment various operational parameters including the depth of electrode penetration into the target skin, the voltage level to be applied to electrode, the power to be transmitted to electrode, the duration over which an electrical signal is to be emitted to electrode, and the delay time during which electrical signal is not to be emitted to an electrode, may be set preliminarily.

Further in an embodiment, the system may include an electrode module that includes an array, which the electrodes may be fixed. In addition, the system may include a motor unit for driving or moving the array including two or more electrodes to penetrate a desired region of skin. It is noted that the system may be used for vessel treatment in various tissue in all anatomical regions in addition to dermatologic regions.

In an embodiment, a system of the present invention may not only allows its users to treat hypervascularity in the dermis or epidermis to be normalized by generating thermal damage thereto or inducing phagocytosis or apoptosis of vascular cell in order to treat melasma, dermal melasma, hyperpigmentation, hypopigmentation, rosacea, flushing, erythema, or telangiectasia, but also to accurately control the degree or level of thermal injury delivered to a vessel directly via an electrode inserted in the skin. Applying heat selectively to a vessel in the skin, instead of applying heat to the entire skin, may reduce the adverse effects caused by overheating of the skin, such as burns, which are common in conventional treatments for melasma, dermal melasma, hyperpigmentation, hypopigmentation, rosacea, flushing, erythema, or telangiectasia. Eliminating or removing a vessel in the skin may also lower the rate of re-occurrence associated with conventional treatments for melasma, dermal melasma, hyperpigmentation, hypopigmentation, rosacea, flushing, erythema, or telangiectasia.

Hereinafter, the present invention will be described with reference to the drawings. Like elements in the drawings are represented by the same reference numerals when possible. In the following description, well-known functions or unnecessary explanation about configurations are not described in detail since they would obscure the subject matter of the present invention.

According to recent studies conducted by the present inventor, lesions of melasma, dermal melasma, hyperpigmentation, hypopigmentation, rosacea, flushing, erythema, or telangiectasia, as well as hair loss, hair removal, excessive sebaceous gland secretion, excessive sweating, and axillary osmidrosis, are related to the number, size, shape, and function of vessels therein. For example, melasma, pigmentation, rosacea, flushing, and telangiectasia lesions clinically and histologically show significant increases in the number and size of blood vessels in the dermis or epidermis, compared to nearby normal skin.

The inventor notes that altering blood vessels in these lesions according to treatment purpose has been found to help to treat those states thereof. In addition, the inventor notes that hair loss may be treated by increasing blood circulation to hair follicles lacking blood supply, while excessive sebaceous gland secretion and excessive sweating may be treated by diminishing abnormal increases in the number and size of vascular structures to target glands.

The inventor also notes that while rosacea, flushing, erythema, and telangiectasia are generally known to be symptoms of underlying vascular problems, melasma, dermal melasma, hyperpigmentation, and hypopigmentation lesions are closely related thereto. Moreover, the inventor has noted excessive development or deterioration of blood vessels that supply nutrition to structures in skin important to hair loss, hair removal, excessive sebaceous gland secretion, excessive sweating, and axillary osmidrosis.

Accordingly, the present invention has been developed to transmit electrical signals to vessels in skin through invasive electrodes for selective treatment of vessels including blood vessels. The inventor notes that blood vessels have a particularly higher impedance relative to the surrounding skin tissue enabling selective treatment of some tissue in skin due to the differences of tissue impedance, conductivity, and dielectric permittivity between individual layers of skin tissue, vessels, and appendages.

The present invention can control the intensity of electric signals that are emitted in electrodes in order to control degree of thermal reaction induced on nearby tissue including vessels. This may allow user to selectively achieve various desired effects including congestion, regeneration, remodeling, growth, regrowth, degradation, or degeneration of vascular structures in skin. Accordingly, a system of the present invention may be employed to treat melasma, dermal melasma, hyperpigmentation, hypopigmentation, rosacea, flushing, erythema, telangiectasia, hair loss, or enable hair removal via skin vessel treatment. Additionally, embodiments of the present invention may be used to treat excessive sebaceous gland secretion(hyperseborrhea), excessive sweating(hyperhidrosis), or related disorders, such as axillary osmidrosis.

Further, embodiments of the present invention may enable a user to effectively treat hyper- or hypovascularity in dermis, epidermis, or skin appendage by generating optimal thermal effects on target vessel therein, or by inducing phagocytosis or apoptosis of target vascular cells. Embodiments of the present invention may also be employed to prevent or reduce common side effects of too much heat being applied to vessel, or reduce recurrence rate of lesion caused by rebounded hyperplasia of removed vessel after conventional treatments.

Embodiments of the present invention may provide a system for treating melanocyte or basement membrane in skin by improving pathological structure or function, or a system for affecting on amount or function of vascular endothelial growth factor (VEGF), which is derived from vessels. As noted conventional invasive high-frequency devices may be limited to coagulating skin with heat for primary purpose of neocollagenesis, hemostasis, and cauterizing vessel directly with high heat. Unlike conventional devices, the present invention encompasses an system for treating vessel in skin by forming electric field within skin. In an embodiment, the system forms an electric field via two or more electrodes that penetrate skin.

In an embodiment an electric field may be uniformly formed in skin via an electrical signal emitted therein to induce thermal effect on blood vessel first rather than surrounding skin tissue. Thus, unlike the direct electrocauterization of blood vessels, an electric signal that forms a uniform electric field may facilitate selective heating of blood vessels, enabling the treatment of various lesions caused by abnormal vascularity. While conventional invasive high-frequency devices which are aimed at stimulating collagen production in skin require the application of a relatively long conduction time, embodiments of the system of the present invention may utilize or generate much shorter conduction time. Moreover, in addition to conduction time, embodiments of the system of the present invention may control other treatment parameters, including signal voltage, power, impedance in skin, etc. as appropriate.

As noted due to their high conductivity, blood vessels may be coagulated first before epidermis or dermis is coagulated via embodiments of the present invention. In an embodiment, the shortest signal conduction times employed by system to selectively coagulate blood vessel may be 50 msec, more generally within 100 msec, or even up to within 300 msec.

In order to prevent excessive thermal damage to non-targeted tissue or to concentrate the desired thermal effect on a target vessel, an electrical signal may have one or more pulses (repeated) with a conduction time and delay time (for example, 5-100 msec) before the next pulse. By employing a repetitive electrical signal with delay time, the applied voltage may be increased, allowing for a greater distance between electrodes (153). Also, the greater voltage (of an applied signal) can increase the degree of thermal effect on blood vessels via a shorter conduction time, thereby minimizing unwanted injury to surrounding tissue due to their lower resistance as noted.

In an embodiment, delay time (between active pulses) may allow for a greater applied voltage, which may induce a faster reaction from blood vessel, thereby preventing/limiting undesired thermal injury to surrounding tissue that may likely occur from sustained delivery of electrical energy. In an embodiment, a conduction time of a repetitive electrical signal may be varied according to configuration of parameters of the system, where the signal conduction time may be set to be the same or different in each cycle. Also, the length of delay time (off duty cycle) may also be varied in the same manner.

Additionally in an embodiment, the conduction time may be varied as a function of the electrical signal's set voltage, electric power, and electric current, as well as the size, thickness, and quantity of target vessels in skin. Further, the electrode insertion depth and thickness, distance between electrodes, and deployment of electrodes relative to the target vessel may also vary the conduction time. In an embodiment, the electrode may be arranged to deliver electrical signals in a bipolar configuration that may be employed in all cases of vessel treatment, except for direct electrocautery of blood vessel.

As noted, melasma, dermal melasma, pigmentation, rosacea, flushing, or telangiectasia lesions clinically and histologically show significant increases in the number and size of blood vessels in dermis or epidermis, compared to normal peripheral skin. The vascular changes apparent in melasma, dermal melasma, pigmentation, rosacea, flushing, or telangiectasia may be due to angiogenesis or vasodilation in association with VEGF(Vascular Endothelial Growth Factor) production. In melasma, dermal melasma, pigmentation, rosacea, flushing, or telangiectasia, abnormal vascularity in the lesion may act as a main reason of incidence or aggravation, because the number of blood vessel distributed throughout the lesions has been shown to be significantly related to the amount of pigment therein. In an embodiment, a system may be employed to reduce target vessel including neovascularization, vessel dilation, or hypervascularity that causes the above conditions.

FIG. 2 is a simplified block diagram of a system 100 for generating electrical signals to treat vessels in skin according to various embodiments of the present invention. As shown in FIG. 2, the treatment system (100) for generating electrical signal with which to treat vessels in skin, according to an embodiment of the present invention, may include a power supply unit (110), an electrical signal generator (130), an electrode module (150), a motor unit (170), a user interface for parameter input (180), and a central processing unit (190).

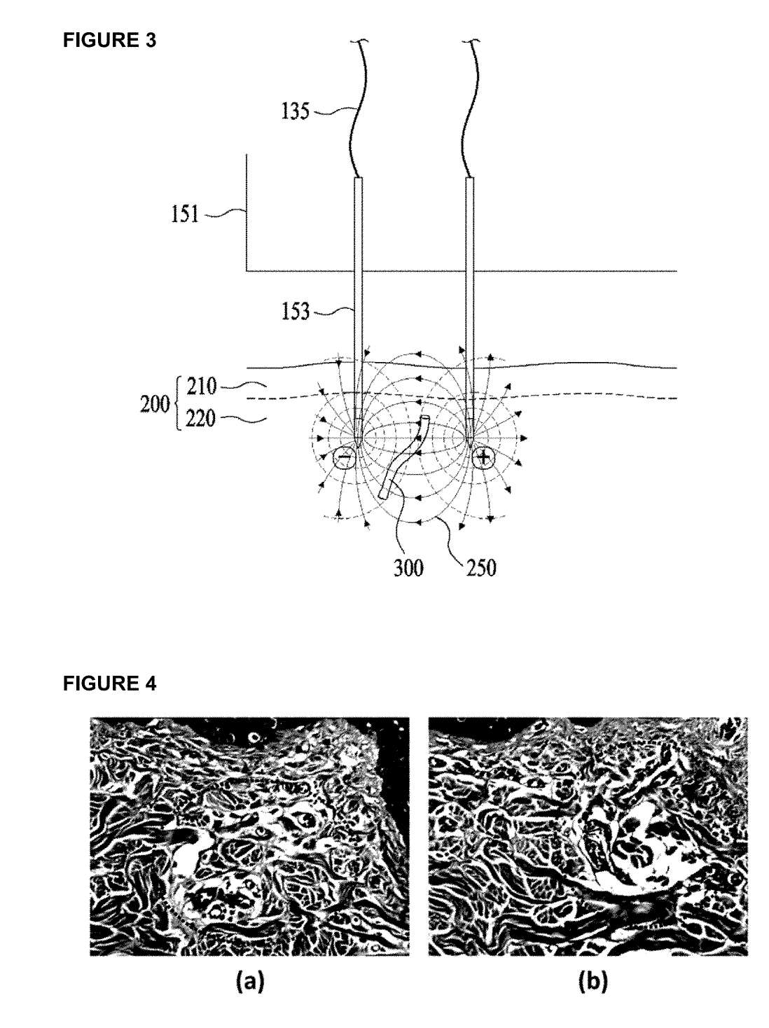

The electrode module (150) may include a fixed portion (FIG. 3, 151) to which two or more electrodes (FIG. 3, 153) are attached. The power supply unit (110) may supply power from outside the system 100 to the electrical signal generator (130) and other components. The electrical signal generator (130) may deliver/generate electrical signals that are conducted by one or more electrode (153) of the electrode module (150) via an electrically conductive material, such as an electrical signal transmission line (FIG. 3, 135). In an embodiment, an electrode (153) may also be connectable directly or indirectly with the electrical signal generator (130) or the motor unit (170), omitting an electrode module (150). In addition in an embodiment, either the electrode (153) or the electrode module (150) may be directly or indirectly connectable electrically to the electrical signal generator (130). In an embodiment, the electrode (153) may be needle shaped and composed of a conductive material.

In an embodiment, the electrical signal generated by the electrical signal generator (130) may be an electromagnetic wave with a frequency ranging from 300 Hz to 300 GHz. In an embodiment, the electrical signal generator (130) may generate an intermediate frequency, a high frequency, and a radio frequency electromagnetic wave or ultrasonic wave. The electrical signal generated by the electrical signal generator (130) may be emitted in the form of an electromagnetic wave of a predetermined frequency. The electrical signal may be transferred to two or more electrodes (153). Heat may then be generated on or around target vessel (300) by transmission of the electrical signal into the lesioned area of the skin (200) via the electrode(153).

In an embodiment, an operator may insert electrodes (153) directly into the lesioned skin (200) by hand, or electrodes (153) may be attached to the electrode module (150) for insertion. In an embodiment, the system 100 may include a motor unit (170) that may automatically drives/insert the electrode (153) or the electrode module (150) to which the electrode (153) is coupled to a predetermined depth into skin (200, FIG. 3). The motor unit (170) may be connected directly or indirectly to the electrode (153) or electrode module (150).

In an embodiment, the electrodes (153) or electrode module (150) may not be connected to the motor unit (170) before insertion into skin (200). It may be inserted into skin (200) by pressing or attaching the electrodes (153) or the electrode module (150) to the motor unit (170) prior the electrode penetration phase. A motor unit (170) may transmit directly or indirectly the force that drives two or more electrodes (153) to penetrate into the skin (200) to a predetermined depth (approximately 1 mm to 2.5 mm in an embodiment). In an embodiment, two or more electrodes (153) may be inserted into the skin (200) so they penetrate the epidermal layer (210) at a predetermined depth of approximately 0.2 mm to 1 mm or the dermal layer (220) at a predetermined depth of approximately 1 mm to 4 mm. The insertion depth of the electrode (153) into target skin (200) may range from 0.2 mm to 4 mm as a function of the patient in an embodiment.

It is noted in an embodiment that it may also be possible to treat a target vessel by placing electrodes (153) on the surface of the skin (200) versus penetrating into skin (200). As described above, the depth of electrode (153) insertion into target skin (200) may be within 4 mm. However, the depth of insertion of electrodes (153) into skin (200) may be deeper than 4 mm in targeted area where skin thickness is relatively thicker and could correspond to the entire thickness of the skin (200) layer. In an embodiment, electrodes (153) or the electrode module (150) may be moved linearly via the motor unit (170), where the motor unit 170 may include an actuator, a motor, a linear motor, a stepping motor, an electromagnet, or a piezoelectric element.

In an embodiment, a user via the user interface (180) may able set several parameters related to the system 100 including the magnitude of the signal voltage, current, the resistance value, the impedance of the target tissue, and the electrical conduction time, and the depth of electrode 153 insertion into skin 200. Upon receiving a control command from the user interface (180), the central processing unit (190) may direct the electrical signal generator (130) to deliver/generate signals having the desired parameters to two or more electrodes (153), in an embodiment, or based on the parameters including electrical current, the resistance value, the impedance, or the electrical conduction time to control the energy amount of the electrical signal.

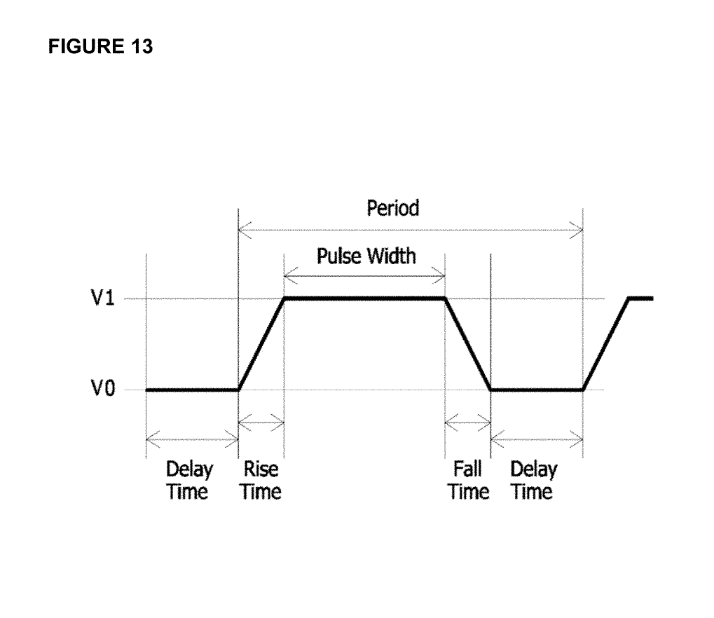

Additionally, the central processing unit (190) may control the power supply unit (110) so that it repeatedly supplies power to the electrical signal generator (130) over a predetermined time interval. FIG. 13 is a simplified diagram of a waveform of an electrical signal that may be generated by the system 100 for generating electrical signals to treat vessels in skin 200 according to various embodiments of the present invention. In an embodiment, the central processing unit (190) may control the electrical signal generator (130) such that it generates an electrical signal repeatedly at predetermined time interval as shown in FIG. 13.

In an embodiment, the applied voltage (V1, FIG. 13) may be in the range of 10 volts to 400 volts (preferably 20 to 300 volts), based on a 100-ohm load. A delay time (or off duty cycle) may be in the range of 0.1 msec (millisecond) to 500 msec (preferably 5 to 300 msec). A conduction time (on duty cycle) may be in the range of 1 msec to 450 msec (preferably 5 to 300 msec). In an embodiment, if the delay time is too short (less than 0.1 msec), undesired thermal damage may occur in non-target tissues (other than target vessel (300)), whereas too long of a delay time (longer than 500 msec) would be too long to induce a enough thermal response on target vessel (300). Conversely, if the conduction time is too long (greater than 450 msec), excessive heat may be generated on tissues other than the target vessel (300, FIG. 3). Similarly, if the conduction time is too short (less than 1 msec), the thermal response elicited or generated in target vessel (300) may be insufficient to provide the desired effect or treatment.

In an embodiment, the total treatment time (equal to the number of repeated cycles of the electrical signal conduction and the delay times) may be closely related to the selected system parameters including the applied voltage, electric power, conduction time, and delay time. If treatment time is too short (too few a number of the cycles), the system 100 may fail to generate a sufficient, desired thermal response, whereas too long a treatment time (too many cycles) may cause excessive, undesired thermal damage to other tissues and the target vessel (300). In an embodiment, a desired therapeutic effect may achieved via the application of a pulsed signal as shown in FIG. 13, in particular with a pulsed signal having relatively high output voltage versus a lower output voltage signal that is continuously applied.

Accordingly, the system 100 may engage the electrical signal generator (130) to generate a pulse type of electrical signal. In an embodiment, the pulsed signal may be alternating current (A.C.) versus a direct current (D.C.) pulse to generate a desired thermal response. In an embodiment, when two electrodes 153 are inserted into the skin around a target vessel (300) as shown in FIG. 3, the signal applied to the electrodes (153) may form a bipolar configuration (one electrode acting as an anode, the other as the cathode. Further in an embodiment, an Alternating Current Pulsed-typed Electric Field may be formed in the skin, by applying an A.C. pulsed electrical signal via the electrode (153) inserted into skin where the electrodes form a bipolar pair and the use of an AC signal causes the electrode polarity to switch periodically, i.e, the anode becomes the cathode, and the cathode becomes the anode in a repeated pattern.

In an embodiment, the signal applied to the electrode pair 153 may be a high-frequency pulsed AC polarity signal, which may be much more effective at vibrating water molecules in skin than a high frequency pulsed D.C. polarity signal and, thereby, generating a greater desired thermal response in target vessels (300). In an embodiment, the use of an alternating polarity, high-frequency pulsed signal (of A.C. polarity) may allow for generating selective thermal response to target vessel (300), compared to the use of a non-alternating polarity high-frequency pulsed signal (of D.C. polarity).

Accordingly, in the present invention, the electric field (250) formed between two electrodes (153) inserted around target vessel (300) as shown in FIG. 3 may be preferably alternate or be an alternating current (AC) electric field due to the application a high-frequency pulsed AC polarity signal to the electrode 153 via the electrical signal generator 130. In an embodiment, when a high output voltage is applied to the electrodes 153 operating a bipolar configuration, a uniform and strong electric field may be formed in a wider area, compared to when a low output voltage is applied to the same electrodes 153. In an embodiment, the thermal response elicited on target vessel (300) may occur more rapidly in the presence of an electric field generated from a high output voltage signal. In such an embodiment the signal pulse width (conduction time) may be made smaller when a high output voltage signal is employed.

As noted in an embodiment, a user may be able to set/select the insertion depth of electrodes (153) into skin (200) via the user interface (180). In an embodiment, the central processing unit (190) may control the degree to which the motor (170) moves the electrode (153) to ensure the electrodes 153 are inserted to the desired depth. As also noted in an embodiment, a user via the user interface (180) may input system parameters including the signal voltage, power, and conduction time and set the insertion depth for electrodes (153). In an embodiment, the central processing unit (190) may control the electrical signal generator (130) so it generates the predetermined or desired electrical signal. In an embodiment, an A.C. or D.C. signal received from the power supply unit (110) may be converted into a predetermined/desired electric signal by the electrical signal generator (130). The resultant electrical signal may be transmitted/applied to electrodes (153).

As noted in an embodiment, the central processing unit (190) may control the motor unit (170) to drive or move individual electrodes (153) or the electrode module (150) into the skin (200) to a desired location or depth according to the electrode (153) insertion depth set by the user through the user interface (180). FIG. 3 is a simplified illustration of a mechanism of vessel (300) treatment via a system (100) delivering electrical signals according to various embodiments of the present invention. As drawn in FIG. 3, skin (200) may include an epidermal layer (210) and a dermal layer (220). In an embodiment, the dermal layer (220) is primarily where target vessel (300) resides that gives rise to melasma, dermal melasma, pigmentation lesion, rosacea, flushing, and telangiectasia.

In another embodiment, a target vessel (300) may be distributed in the epidermal layer (210), while in a further embodiment, a target vessel (300) may be distributed throughout both the epidermal layer (210) and the dermal layer (220). Accordingly, electrodes (153) may be inserted within the epidermal layer (210) only or further into the dermal layer (220) as needed, depending on the distribution of target vessel (300) throughout the skin.

In an embodiment, vessels (300) that give rise to melasma, dermal melasma, hyperpigmentation, hypopigmentation, rosacea, flushing, erythema, or telangiectasia lesions are referred to target vessel (300). As shown in FIG. 3, when target vessel (300) are present only in the dermal layer (220), it may be preferable that the inserted electrodes (153) penetrate the dermal layer (220). In an embodiment, however electrodes (153) may be inserted such that they penetrate only the epidermal layer (210) with appropriate energy setting (voltage, power, and conduction time) and still effectively treat the target vessel. As noted in an embodiment, it may be possible to generate a sufficient thermal response in a target vessel (300) via electrodes (153) placed on the surface of the skin.

For treating melasma, dermal melasma, pigmentation, rosacea, flushing, or telangiectasia lesions in the dermal layer (220), electrodes (153) may be inserted into the dermal layer (220) among target vessels (300) within the lesioned area of the skin in an embodiment. In an embodiment, two electrodes (153) may be inserted into the skin about a target vessel (300) (positioned between two electrodes (153)). In such an embodiment, an electric field (250), as shown in FIG. 3, may be formed between the two electrodes (153) as a function of the applied signal. In an embodiment, a target vessel (300) may still receive desired thermal exposure or have a desired thermal response when located deeper than the electrode (153) can penetrate or positioned immediately beneath the electrode (153), as long as the target vessels (300) are located within the electric field (250) formed between the electrodes (153).

In an embodiment, the electrodes (153) may be inserted into the skin with target vessel (300) positioned in an A.C. electric field (250) formed between two electrodes (153) based on application of an AC, high frequency, pulsed signal. Such an embodiment may conduct a high-frequency signal of A.C. polarity between two electrodes (153) operating in a bipolar configuration to induce a selective/desired thermal response in target vessel (300) positioned between the two, bipolar electrodes (153). In an embodiment, such a treatment may limit treatment to the vessel 300 while sparing injury to surrounding tissue. In an embodiment, it may be preferable that the high-frequency signal applied to the electrode (153) has a main operating frequency between 0.1 MHz and 100 MHz.

It is noted that in some instances a target vessel (300) may be positionable relatively close or adjacent to one or more electrodes (153) while other times a target vessel (300) may be relatively far from one or more electrode (153). In such embodiments, a user may change the type of electrical signal or adjust the voltage, power, or conduction time of the electrical signal applied to the electrodes 153 based on their distance from a target vessel (300).

As noted and show in FIG. 3 in an embodiment electrode (153) may be inserted into skin (200) and receive electrical signals via the electrical signal transmission line (135) causing the electrical signal transmitted to the inserted portion of the electrode (153) to be emitted to the blood vessel (300). In an embodiment, the electrical signal transmission line (135) may be connected directly to the electrode (153) or indirectly via a printed circuit board, a solder, an electrical pin (a pin that is capable of conducting electric power and being bent and stretched), a pogo pin, an electric conduction plate, an electric conduction rod, or an electrical connector to transmit electrical signal (not shown).

In an embodiment, electrical signals applied to a lesion may be concentrated to target vessel (300) in the skin, generating heat in the target region, which heat treat target vessel (300) and thus form a therapeutic mechanism. As noted in an embodiment system (100) may be employed to deliver electrical signals to electrode (153) inserted into the skin or in contact with the skin surface in order to generate thermal injury on or around target vessel (300) in the skin. Through clinical experiments with the treatment system (100) for generating electrical signal with which to treat vessel in the skin, the inventor notes that the causative target vessel (300) that give rise to lesions, such as melasma, dermal melasma, pigmentation, rosacea, flushing, or telangiectasia, are mostly neovascular (premature blood vessel), and the binding between the cells that constitute their vessel walls is in a loose state, relative to normal vessels. Moreover, the thickness of the vessel wall for neovasculature is thinner than that for normal blood vessels, and the cellular structures in the vessel wall are weak. Thus, unlike normal blood vessels, these immature blood vessels can be easily destroyed by relatively weak electrical stimulation.

Accordingly, by applying electrical signals via the system 100 to destroy the causative, target vessel (300) that give rise to lesions, such as melasma, dermal melasma, pigmentation, rosacea, flushing, or telangiectasia, treatment for these conditions may be possible although not simple. To induce suitable damage on target vessel (300), an electrical signal that is neither too weak nor too strong are required to be applied via the system 100. Therefore, in an embodiment delicate control of the electrical signal generator (130) is required. Since target vessel (300) of neovascularity may be treated with relatively weak signals employment of strong electrical signals may result in artificial stimulation, cauterization, elimination, or severe destruction, and compensation mechanisms that may quickly give rise to more new vessels due to a phenomenon known as vascular hyperplasia.

Consequently, treatment methods aimed at simply destroying the causative, target vessel (300) in melasma, dermal melasma, pigmentation, rosacea, flushing, or telangiectasia lesions via electrical signal of random strength may result in treatment outcomes of vascular hyperplasia or post-inflammatory hyperpigmentation (PIH), a condition that further exacerbates lesions of melasma, dermal melasma, pigmentation, rosacea, flushing, or telangiectasia.

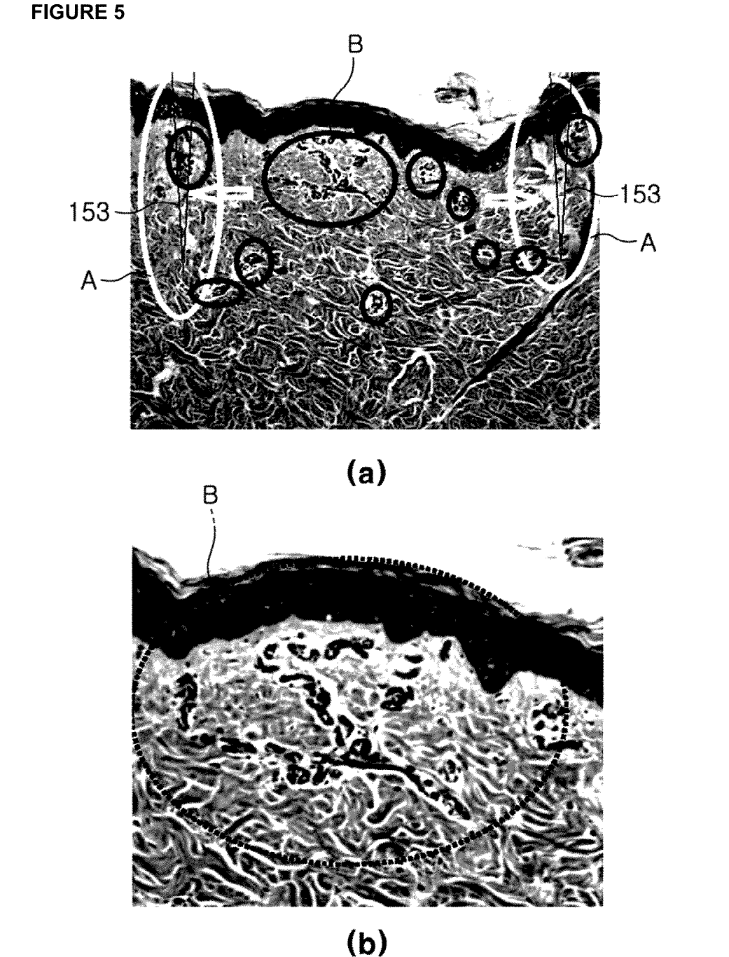

As shown in FIGS. 4 and 5, when the therapeutic system (100) of the present invention is used to apply electric signals to the skin of a micro pig, vessels in dermal layer of skin responded selectively with the signal. No evidence that the blood vessels were destroyed or that excessive bleeding occurred was recorded. In particular, in FIG. 5(a), heat-damaged tissue (pale pink in color) is observed in regions demarcated by white ovals labelled region A, around which electrodes (153) have been inserted. The black circles labelled as region B highlight areas of selective thermal damage to the walls of vessels (pale pink in color) neighboring the target vessel (300) (the largest black circle). FIG. 5(b) is an enlarged photograph of the target vessel (300) in region B of FIG. 5(a). The photograph more clearly exhibits a selective thermal response along the vessel wall (pale pink color).

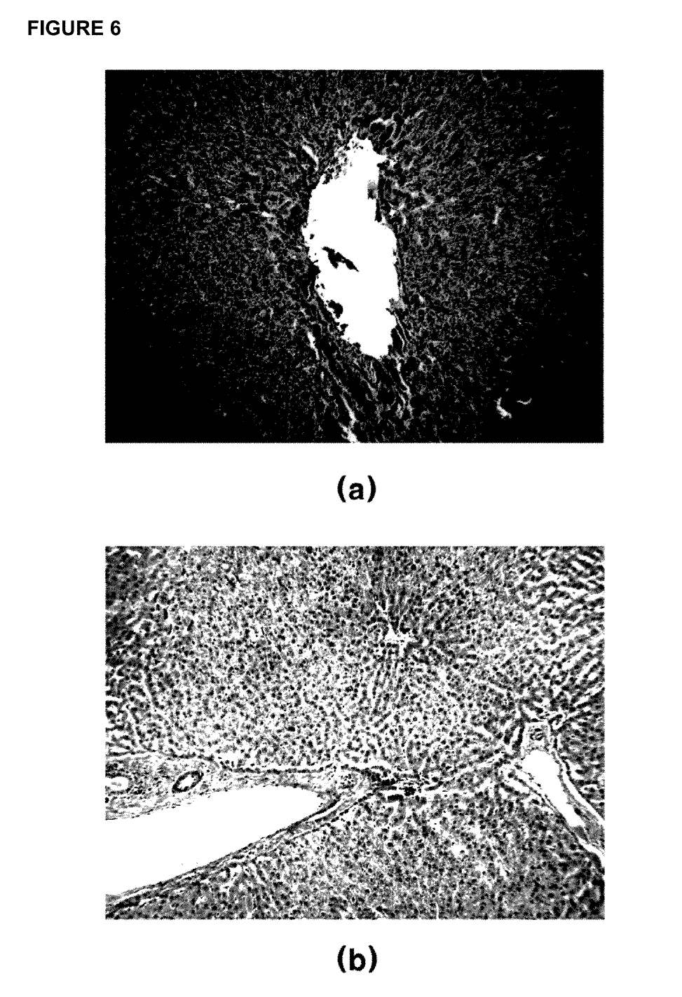

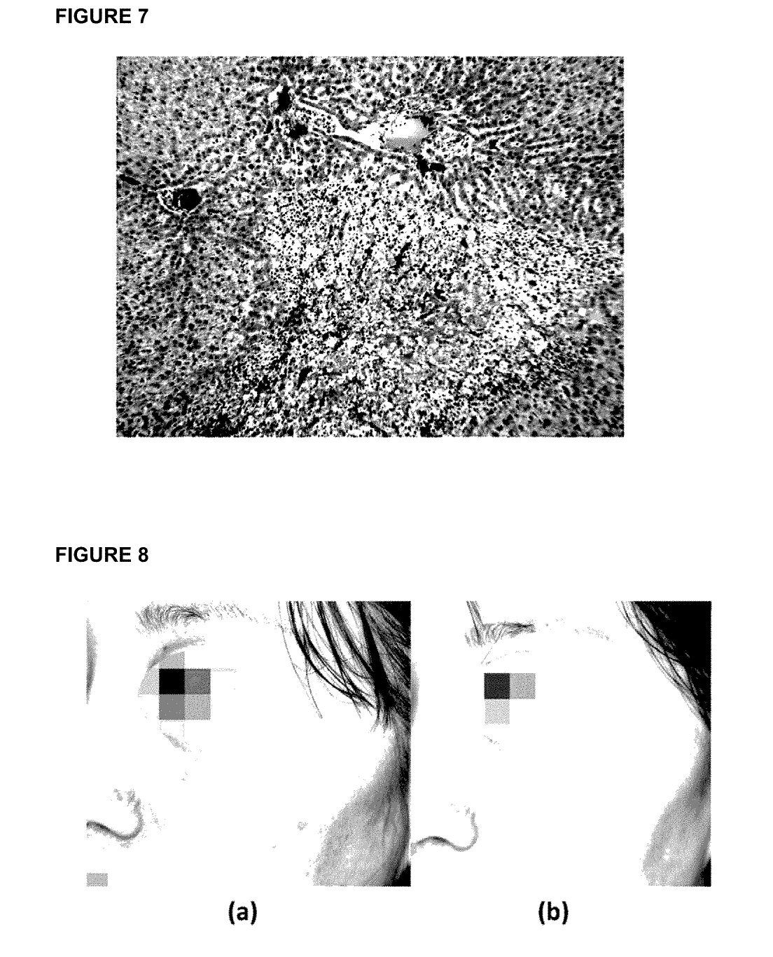

As shown in the figures, no thermal damage occurs in skin tissue between the two electrodes (153) in regions A (area around the electrodes), except for area surrounding the target vessel (300) in region B due to application of energy to electrodes (153) via system (100). FIGS. 6 and 7 shows experimental results from treating bovine liver tissue with the treatment system (100). Liver tissue was selected in order to more clearly show the selective thermal response on blood vessels, since liver tissue, in comparison with the skin, is composed primarily of hepatocytes and blood vessels and has relatively uniform tissue impedance, conductivity, and permittivity. Employing the system (100) to treat the liver tissue confirmed that the embodiments of the present invention may be employed to deliver electrical signal that selectively reacts with vessel in bovine tissue.

In addition, the electrical signal generated by system 100 mainly induced a thermal reaction along the outer surface of the vessel where the electrical signals were conducted along the vessel wall. Any tissue changes elicited by the thermal reaction induced by electrical signal were mainly observed in the tunica adventitia of blood vessel, while the tunica intima and tunica media were preserved. As stated above, non-selective aggressive destruction of vessels in a lesion may stimulate excessive production of Vascular Endothelial Growth Factor (VEGF) and promotes blood vessel regeneration by Vascular Hyperplasia, leading to worsening of the lesion.

However, a selective thermal reaction, such as that induced by an embodiment of the present invention, along target vessel (300) or the outer layer of target vessel (300) may promote the regeneration of target vessel (300) in lesioned skin into normal vessel structure. Moreover, inducing a selective thermal reaction may decrease the risk of side effects that may occur as the results of excessive nonselective damage to vascular and dermal structures and it improved clinically in melasma, dermal melasma, pigmentation, rosacea, flushing, or telangiectasia lesions.

In clinical experiments via the treatment system (100) for generating electrical signal with which to treat vessel in the skin, the treatment of melasma, dermal melasma, pigmentation, rosacea, flushing, or telangiectasia lesions, appropriate thermal damage to endothelial cell in vessel wall may restore abnormal vascular hyperplasia to normal, or normalizes the increased VEGF amount or levels, thereby promoting angiogenesis, and returning dilated vessel to normal size. Appropriate thermal damage to endothelial cells may also induce the phagocytosis or apoptosis thereof, enhancing the therapeutic effect vessel treatment.

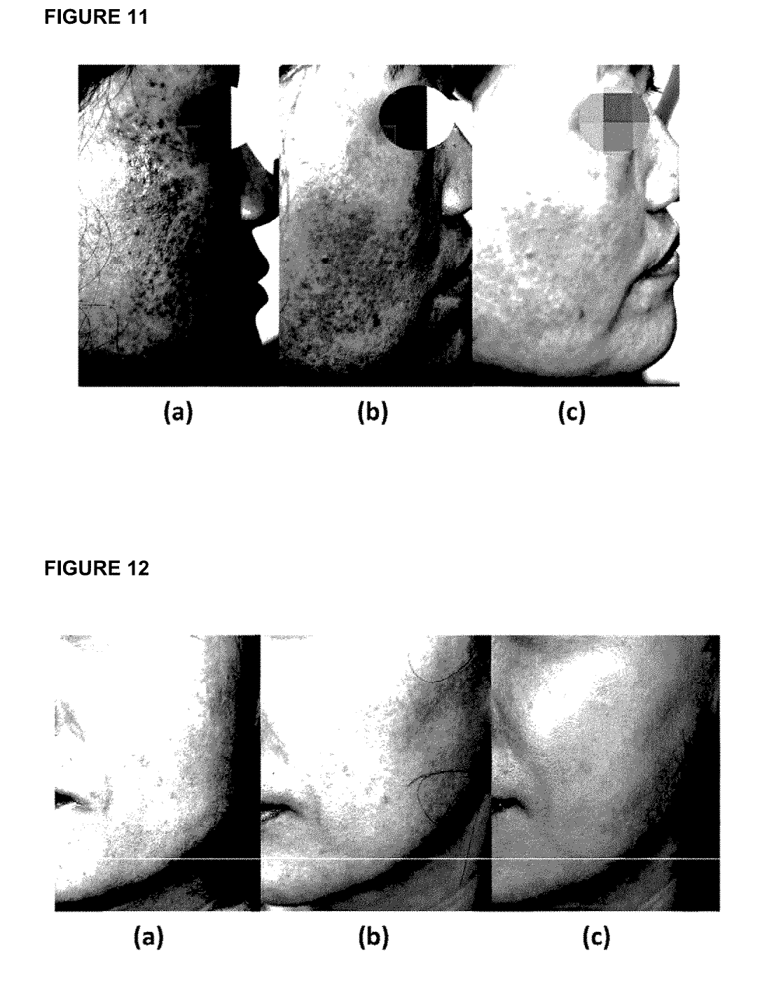

As shown in FIGS. 11 and 12, a vessel removed by the mechanism of inducing voluntary phagocytosis or apoptosis of endothelial cell show a markedly lower rate of recurrence, which was confirmed in clinical study. The technical principle of generating appropriate thermal damage to endothelial cell that constitute the causative, target vessel (300) that give rise to melasma, dermal melasma, pigmentation, rosacea, flushing, or telangiectasia lesions via an embodiment of the present invention will now be described in more detail.

Most of the blood in target vessels (300) that cause melasma, dermal melasma, pigmentation, rosacea, flushing, or telangiectasia is composed of water, accordingly the electrical conductivity of such a vessel in an electric field may be higher than that of surrounding tissue. Thus, such a vessel strongly may attract most of the electrical current delivered to the skin in an embodiment. Also, vessel walls may exhibit much higher difference of impedance and permittivity from blood, and thus, in the presence of an electric field, the ionics that are located at the vessel wall may vibrate, causing a thermal reaction: electrical signal in the form of electromagnetic waves may cause water molecules and ionics to vibrate, thereby generating friction and heat.

Further, electric signals may exit vessels through blood that is in contact with the inner vessel wall, and heat may be dispersed by blood flowing through the vessel. Thus, a thermal reaction may be less likely to occur inside the vessel wall. Instead, heat may be concentrated on the outer vessel wall. If the conduction time of the electrical signal is further increased, more heat may be generated as a whole, although this can cause excessive damage to the blood vessel. Moreover, the heat generated in the vessel wall may cause heat damage to endothelial cell.

In accordance with the above, a treatment system (100) may be capable of eliciting a therapeutic thermal response concentrated on and around the vessel wall of causative, target vessels (300) that give rise to melasma, dermal melasma, pigmentation, rosacea, flushing, or telangiectasia lesions, without causing injury to surrounding tissue. Accordingly, by generating thermal damage to vessels in the skin with electrical signal, the treatment system (100) may induce selective thermal injury to endothelial cell of target vessel (300) and may generate frictional heat stemming from the vibration of water and ionic substances (or electrolytes) in target vessel (300). The treatment system (100) may be used for normalizing abnormal hyperplasia of endothelial cell or for normalizing increases in VEGF in treatment of conditions exhibiting angiogenesis or vasodilation. As is stated above, the treatment system (100) also may also induce phagocytosis or apoptosis of endothelial cell to achieve a desired therapeutic effect.

In order to induce phagocytosis or apoptosis of endothelial cell, an appropriate degree of thermal injury should be applied to endothelial cell. The results of repeated clinical experiments to find the optimal conditions for generating a sufficient thermal response using the treatment system (100) are shown in Table 1.

TABLE 1

| | Condition 1 | Condition 2 | Condition 3 |
|---|---|---|---|
| Voltage (Vrms: 100Ω loaded) | 5~400 | 10~300 | 20~250 |
| Power (W: 100Ω loaded) | 0.25~1,600 | 1.0~900 | 4.0~625 |
| Conduction time (msec) | 1~500 | 5~400 | 10~300 |

The voltage and power values in Table 1 are measured values for when the treatment apparatus (100) is applied with a load resistance of 100 Ω (Ohm; resistance). In other words, the voltage (Vrms unit: Volts) and power values are those at a load resistance of 100Ω (Ohm; resistance) for electrical signals delivered to electrodes (153) inserted in the skin (200) (epidermal layer [210] and dermal layer [220]). The conduction time (msec unit: 0.001 sec) in Table 1 refers to the time over which electrical signal was applied to electrode (153) inserted into the skin (200). More specifically, the conduction time was measured as the time for delivering an electric signal to the skin (200) during one shot.

An embodiment of the system 100 may be employed with one or more of the parameter values shown in Table 1 via the user interface (180). The central processing unit (190) may then control the delivery of electrical signals according in the selected parameters. Using the treatment conditions in Table 1 in skin via system 100, melasma, dermal melasma, pigmentation, rosacea, flushing, or telangiectasia lesions were improved, but such conditions showed greater improvement when using the narrower treatment conditions in condition 2 in skin via system 100.

When the treatment conditions were set to condition 3 in system 100 and applied to skin, compared to condition 2, the degree of apoptosis and treatment effects on lesions were more prominent. Applying the parameters in conditions 2 to 3, the inventor of the present invention conducted clinical tests in humans and animals using the treatment apparatus (100) to treat melasma, dermal melasma, pigmentation, rosacea, flushing, and telangiectasia lesions. The results will now be described with reference to FIGS. 4 to 12.

FIG. 4 presents photographs of results from animal experiments on micro pig skin. Comparing results (a) before treatment and those (b) after treatment, vessel responses to electrical signal were visible in the dermis. There were no signs that the vessels were destroyed or that excessive bleeding occurred. Meanwhile, damage to vascular cells could be confirmed. FIGS. 6 and 7 are photographs of results from animal experiments on bovine liver tissue immediately after application of electrical signal via system 100.

As seen in FIGS. 6 and 7, an electrical signal induced selective thermal reaction on vessels in the bovine liver tissue. The tissue changes elicited by the thermal reaction induced by electrical signals were mainly observed in the tunica adventitia of blood vessels, while the tunica intima and tunica media layers were preserved. FIG. 8 includes photographs of results from a clinical trial in human skin. Comparing results (a) before treatment and those (b) after 2 months of treatment via system 100 at 1-week intervals, marked improvement in vascular lesions, such as melasma, dermal melasma, pigmentation, rosacea, flushing, and telangiectasia, was noted.

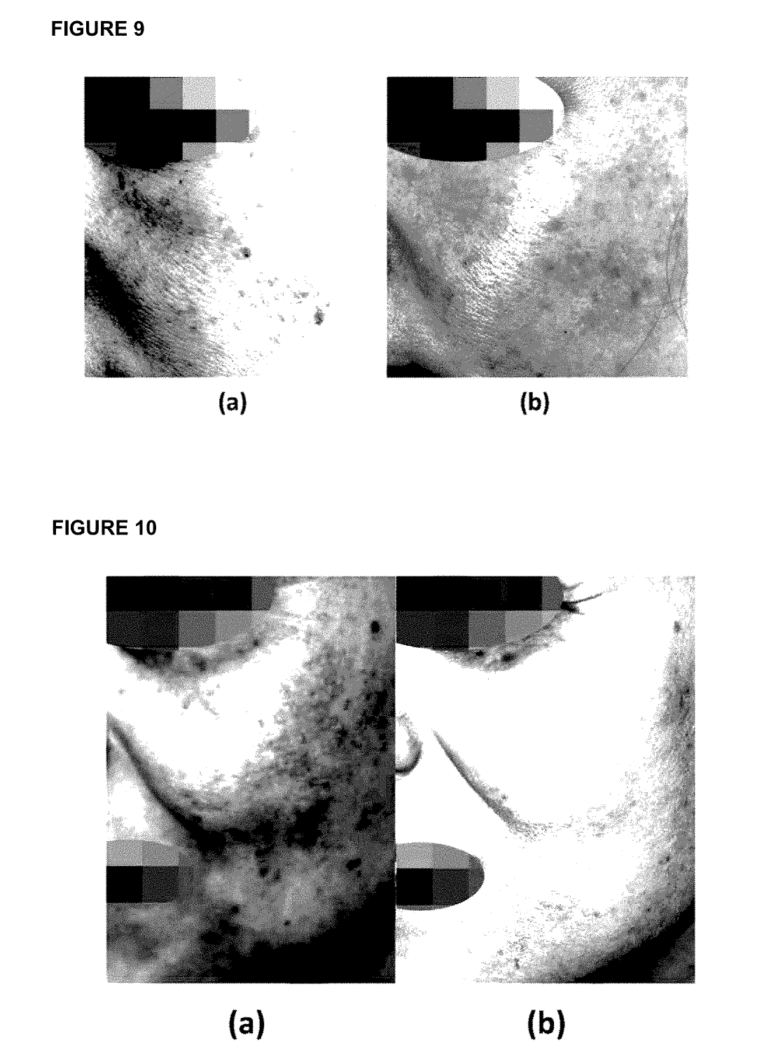

FIG. 9 includes photographs of results from another patient in the same clinical trial. Comparing results (a) before treatment and those (b) after 2 months of treatment using system 100 at 1-week intervals, marked improvement in vascular lesions, such as melasma, dermal melasma, pigmentation, rosacea, flushing, and telangiectasia, was noted. FIG. 10 includes photographs of results from a third patient included in the same clinical trial. Comparing results (a) before treatment and those (b) after 2 months of treatment via system 100 at 1-week intervals, marked improvement in vascular lesions, such as melasma, dermal melasma, pigmentation, rosacea, flushing, and telangiectasia, was noted.

FIG. 11 includes photographs of results from a fourth patient included in the same clinical trial. Comparing results (a) before treatment and those (b) after 2 months of treatment via system 100 at 1-week intervals with those (c) at 1 year after completing the treatment course, marked improvement in vascular lesions, such as melasma, dermal melasma, pigmentation, rosacea, flushing, and telangiectasia, was noted. Moreover, unlike conventional treatment, the appearance of the lesion continued to improve until one year after completion of the treatment, and there were no signs of lesion recurrence.

FIG. 12 includes photographs of results from a fifth patient included in the same clinical trial. Comparing results (a) before treatment and those (b) after 2 months of treatment via system 100 at 1-week intervals with those (c) at 1 year after completing the treatment course, marked improvement in vascular lesions, such as melasma, dermal melasma, pigmentation, rosacea, flushing, and telangiectasia, was noted. Moreover, unlike conventional treatment, the appearance of the lesion continued to improve until one year after completion of the treatment, and there were no signs of lesion recurrence.

In an embodiment, electro thermal responses induced by electrical signal applied to the skin via system 100 may vary according to the resistance of individual tissues. In an embodiment, electrical signals may be delivered to the skin in a monopolar mode via system 100, consisting of an active electrode with negative polarity and a ground electrode with positive polarity, or in a bipolar mode, in which both electrodes 153 are active. In the monopolar mode, an electrical circuit may be formed wherein electric current (electrons) flows from the active electrode through the patient's body to the ground electrode. In the bipolar mode, the flow of the electric current is limited to target tissues. In an embodiment, the bipolar mode may be preferable to the monopolar mode, because the transmission of energy in the bipolar mode may be safer to the human body and may be concentrated on target sites.

In an embodiment, the system 100 may transmit electric signals to the skin via electrode 153 that penetrate into target tissue, enabling it to more precisely control the depth of treatment, compared to conventional, non-invasive methods. Moreover, it may offer more uniform treatment at deeper regions of the skin. Another advantage of system 100 is that the discontinuous emission of energy may allow for a selective tissue thermal reaction to formed in the target tissue. Additionally, while systemic injury to blood vessels can be fatal, the invasive method of the system 100 according to an embodiment may facilitate localized treatment that is relatively safe.

In addition, further research with embodiments of the present invention has indicated that hair roots in the skin are conductors of electric current, particularly the outer sheath, the root muscle, and fibrous connective tissue, similar to the outer walls of vessels. Therefore, the treatment apparatus 100 may help improving hair loss, which is a complicated lesion. Additionally, by adjusting the intensity of electrical signal applied to the hair follicle via embodiments of the present invention permanent hair removal may be achieved. In treatment of melasma, embodiments of the present invention may be combined with conventional treatments, such as LASER toning, drug therapy, etc., in order to increase their therapeutic effects and to further lower the risk of recurrence.

In summary in an embodiment, thermal reactions may be selectively induced only on desired vessel tissue, and unnecessary damage to other surrounding tissues can be prevented or limited. In addition, by conducting electrical signal in a pulsed manner via an embodiment, it may be possible to avoid unnecessarily damaging surrounding tissue or causing excessive damage to vessel tissue, thereby potentially shortening the recovery period after treatment and reducing the risk of side effects. In addition, in an embodiment of the present invention, it may be possible to control the degree of thermal reaction generated on vessel tissue, thereby preventing or limiting bruising, vascular hyperplasia, and PIH, which are caused by excessive damage to blood vessels. Also, in contrast to currently available treatment, embodiments of the present invention utilize penetrating electrodes that allow for more uniform treatment of vascular tissue at deeper regions in the skin.

Further, with embodiments of the present invention, implementation of electrodes 153 in a bipolar configuration may confine the transmission of electrical current to within target lesions, unlike unipolar electrodes with which electrical current is applied to the whole body. Such a configuration is particularly advantageous for treating patients suffering from heart disease or who wear a pacemaker, as they would be contraindicated for treatment with monopolar electrodes. Additionally, with embodiments of the present invention, vascular tissue may be selectively treated and pain caused by the thermal reaction can be reduced, providing a more comfortable procedure for the patient.

It is noted that embodiments of the present invention for use in treating blood vessel is not limited to only application in the dermatology applications. Embodiments of the present invention may also be applied to treat vessels in tissue of a patient, including Gastro Intestinal systems tissue including oral cavity, pharynx, larynx, esophagus, stomach, intestines, anus, liver, spleen, gall bladder, or pancreas, or Respiratory system tissue including trachea, lung, pleura, or chest wall, as well as brain, spinal cord, all neurological system and subcutaneous tissues.

The terminology used herein is for the purpose of describing particular embodiment only and is not intended to be limiting of the invention. The singular forms “a,” “an,” and “the” include plural referents unless the context clearly dictates otherwise. In the present application, the terms “comprises” or “having,” etc. are intended to specify the presence of stated features, integers, steps, operations, elements, components, or combinations thereof, but do not preclude the presence or addition of one or more other features, integers, steps, operations, elements, components, or combinations thereof.

While the present invention has been shown and described with reference to exemplary embodiment thereof, it is to be understood that the invention is not limited to the disclosed exemplary embodiment. It will be understood by those skilled in the art that various changes in form and details may be made therein without departing from the spirit and scope of the present invention.

Embodiments of the present invention are recognized for its industrial applicability in the medical equipment industry.

1. An apparatus for treating a blood vessel in skin, comprising:
at least two electrodes configured to be inserted in skin and positioned about the blood vessel to deliver electromagnetic energy to target the blood vessel; and
an electrical signal generator electrically coupled to the at least two electrodes, and applying an electromagnetic signal across the at least two electrodes having a voltage level between 20 Vrms and 250 Vrms for 10 msec to 300 msec.

2. The apparatus of claim 1, wherein the electrical signal generator repetitively applies an electromagnetic signal across the at least two electrodes for 10 msec to 300 msec followed by at least one delay time.

3. (canceled)

4. The apparatus of claim 1, wherein the electrical signal generator repetitively applies an electromagnetic signal across the at least two electrodes to thermally damage the blood vessel.

5. The apparatus of claim 1, wherein the electrical signal generator repetitively applies an electromagnetic signal across the at least two electrodes to form an electric field of Alternating Current (A.C.) between the at least two electrodes.

6. The apparatus of claim 1, wherein the at least two electrodes form a bipolar configuration.

7. (canceled)

8. The apparatus of claim 1, wherein the apparatus may improve at least one among melasma, dermal melasma, hyperpigmentation, hypopigmentation, rosacea, flushing, erythema, or telangiectasia lesions in the skin about the blood vessel.

9. (canceled)

10. The apparatus of claim 1, wherein the electrical signal generator repetitively applies an electromagnetic signal across the at least two electrodes to thermally damage an outer layer of blood vessel.

11. (canceled)

12. (canceled)

13. (canceled)

14. The apparatus of claim 1, wherein the electrical signal generator applies an electromagnetic signal across the at least two electrodes having a frequency from 0.1 MHz to 100 MHz.

15. (canceled)

16. (canceled)

17. (canceled)

18. An apparatus for treating a blood vessel in skin, comprising:
at least two electrodes configured to be inserted in skin and positioned about the blood vessel to deliver electromagnetic energy to target the blood vessel; and
an electrical signal generator electrically coupled to the at least two electrodes, and creating an electromagnetic wave across the at least two electrodes.

19. The apparatus of claim 18, wherein the electrical signal generator repetitively creates an electromagnetic wave across the at least two electrodes for 10 msec to 300 msec.

20. The apparatus of claim 18, wherein the electrical signal generator repetitively creates an electromagnetic wave across the at least two electrodes for 10 msec to 300 msec followed by at least one delay time.

21. The apparatus of claim 18, wherein the electrical signal generator repetitively creates an electromagnetic wave across the at least two electrodes to thermally damage the blood vessel.

22. The apparatus of claim 21, wherein the at least two electrodes form a bipolar configuration.

23. The apparatus of claim 18, wherein the electrical signal generator repetitively creates an electromagnetic wave across the at least two electrodes to thermally damage an outer layer of the blood vessel.

24. An apparatus for treating a blood vessel in skin, comprising:
at least two electrodes configured to be inserted in skin and positioned about the blood vessel to deliver electromagnetic energy to target the blood vessel; and
an electrical signal generator electrically coupled to the at least two electrodes, and applying a high frequency, alternating current electromagnetic signal across the at least two electrodes.

25. The apparatus of claim 24, wherein the electrical signal generator repetitively applies a high frequency, alternating current electromagnetic signal across the at least two electrodes for 10 msec to 300 msec.

26. The apparatus of claim 24, wherein the electrical signal generator repetitively applies a high frequency, alternating current electromagnetic signal across the at least two electrodes for 10 msec to 300 msec followed by at least one delay time.

27. The apparatus of claim 24, wherein the electrical signal generator repetitively applies a high frequency, alternating current electromagnetic signal across the at least two electrodes to thermally damage the blood vessel.

28. The apparatus of claim 27, wherein the at least two electrodes form a bipolar configuration.

29. The apparatus of claim 24, wherein the electrical signal generator repetitively applies a high frequency, alternating current electromagnetic signal across the at least two electrodes to thermally damage an outer layer of the blood vessel.
